(19) United States
(12) Plant Patent Application Publication
(10) Pub. No.: US 2024/0215470 P1
(43) Pub. Date: Jun. 27, 2024

(54) Agapanthus plant named 'Monsapho'

(50) ;

(71) Applicant: Monrovia Nursery, Azusa, CA (US)

(72) Inventor: Jonathan Pederson, Dayton, OR (US)

(73) Assignee: Monrovia Nursery, Azusa, CA (US)

(21) Appl. No.: 18/087,481

(22) Filed: Dec. 22, 2022

(51) Int. Cl. A01H 6/10

(52) U.S. Cl. PLT/398

(57) ABSTRACT

A new and distinct cultivar of Agapanthus plant named ‘MONSAPHO’ is disclosed, characterized by large, abundant, pure white flowers, sturdy flower stalks, and compact habits. The new variety is an Agapanthus, typically produced as an ornamental plant.

Latin name of the genus and species: Agapanthus hybrid.

Variety denomination: ‘MONSAPHO’.

### BACKGROUND OF THE INVENTION

The new Agapanthus cultivar is a product of a naturally occurring mutation of the unpatented variety Agapanthus ‘Scrarey09’. The new variety was found and selected by the inventor at a nursery in Dayton, Oregon in June 2018.

Asexual reproduction of the new cultivar ‘MONSAPHO’ was first performed in September 2018, at the same nursery in Dayton, Oregon, by tissue culture and division. Subsequent propagation has shown that the unique features of this cultivar are stable and reproduced true to type in successive generations.

### SUMMARY OF THE INVENTION

The cultivar ‘MONSAPHO’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘MONSAPHO’. These characteristics in combination distinguish ‘MONSAPHO’ as a new and distinct Agapanthus cultivar:
1. Very compact habit.
2. Extremely floriferous.
3. Large, pure white flowers.
4. Very sturdy flower stalks held just above foliage.

### PARENT COMPARISON

Plants of the new cultivar ‘MONSAPHO’ are similar to the parent variety in most horticultural characteristics. However, characteristics of the new cultivar differ in the following:
1. Flowers of the new variety are pure white, while flowers of the parent are blue.
2. The new variety is a more compact plant than the parent.
3. Flowers of the new variety are larger and more numerous than flowers of the parent.

### COMMERCIAL COMPARISON

Plants of ‘MONSAPHO’ are similar in some horticultural characteristics to the variety Agapanthus africanus ‘Albus’, unpatented. The new variety, however, differs in the following:
1. Foliage of the new variety is more compact than foliage of this comparator .
2. Flower stems of the new variety are more compact than flower stems of this comparator.
3. The new variety is more floriferous than this comparator.

Plants of ‘MONSAPHO’ are similar in some horticultural characteristics to the variety Agapanthus africanus ‘RFDD’, unpatented. The new variety, however, differs in the following:
1. The new variety has larger, single flowers, while this comparator has smaller, double to semi-double flowers.
2. The new variety is a more robust grower than this comparator.

### BRIEF DESCRIPTION OF THE PHOTOGRAPHS

The accompanying photograph in FIG. 1 illustrates in full color typical plants of ‘MONSAPHO’ grown at a commercial nursery in Dayton, Oregon. These plants are approximately one year old, shown in a one-gallon pot.

FIG. 2 illustrates a closer view of the inflorescence.

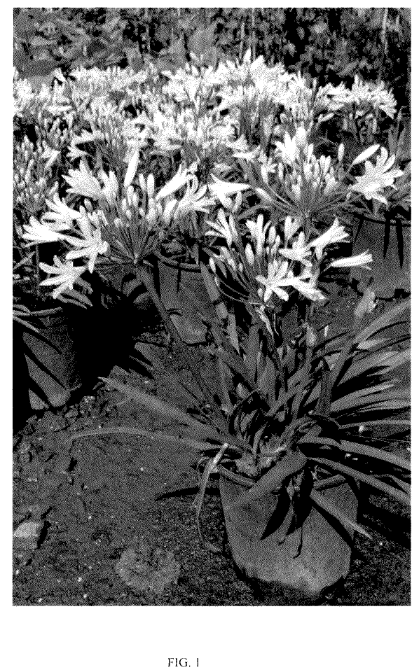

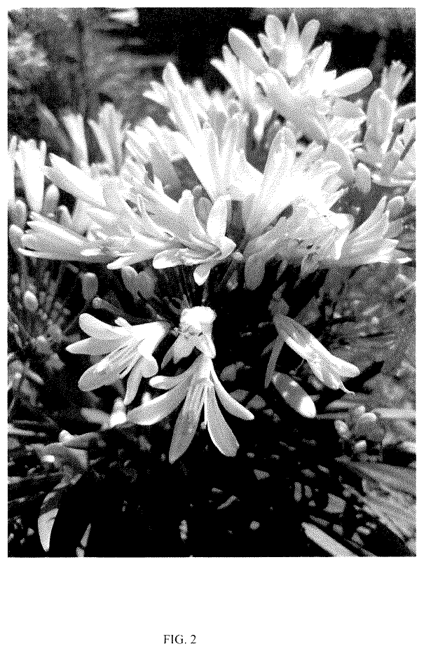

The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007, except where general terms of ordinary dictionary significance are used. The following observations and measurements describe ‘MONSAPHO’ plants grown outdoors in Royal Oaks, California. Plants are approximately 1 year old. Temperatures ranged from approximately 8° C. to 15° C. at night and 10° C. to 32° C. during the day. No artificial light, photoperiodic treatments were given to the plants. No chemical treatments were given to plants.

Measurements and numerical values represent averages of typical plant types.
Botanical classification: Agapanthus hybrid ‘MONSAPHO’.

### PROPAGATION

Propagation method: Tissue Culture.
Root description: Thick, fleshy root system colored near RHS Orange-White 159C.

### PLANT

Age of plant described: 240 days from a rooted cutting.
Growth habit: Clump forming tender perennial with arching, pendulous foliage. Plants evergreen to about −5° C.
Height: Foliar plane 20 to 25 cm. Flowering plane 30 to 35 cm.
Plant spread: Approximately 30 cm.
Growth rate: Moderate.
Diameter of stem: Flower stems 5 to 8 mm.
Growth pattern: The plant makes growth throughout most of the year but particularly in the summer months. This grow is in the form of basal shoots or pups as well as some leaf extension. From mid-summer flowers start to appear and remain for much of the summer.

### FOLIAGE

Leaf:
   Quantity.—Average 20.
   Average length.—9 to 15 cm.
   Average width.—1 cm.
   Shape of blade.—Linear.
   Apex.—Acute.
   Base.—Truncate, non-clasping.
   Margin.—Entire.
   Aspect.—First year growth is mainly upright, slightly upwardly curved. With maturity foliage becomes more arched.
   Texture of top surface.—Smooth.
   Texture of bottom surface.—Smooth.
   Color.—Young foliage upper side: Near RHS Green 143C. Young foliage under side: Near RHS Green 143A. Mature foliage upper side: Near RHS Green 137B. Mature foliage under side: Near RHS Green 137A.

### FLOWER

Natural flowering season: Summer.
Length of flowering period: Approximately 8 weeks.
Inflorescence type: Terminal umbel with approximately 30 individual flowers and 5 to 10 buds.
Individual flower type: Single.
Flower profile: Funnelform.
Lasting quality: Inflorescence shows no significant signs of aging for approximately 5 to 6 weeks.
Bud: Measured at approximately 2 to 5 days from opening.
   Shape.—Narrow ovate.
   Length.—Approximately 1.5 cm.
   Diameter.—Approximately 0.7 cm.
   Color.—Near RHS Green-White 157C.
Umbel size:
   Diameter.—Average 15 cm.
   Height.—Average 10 cm.
Corolla:
   Arrangement.—Apetalous, single, campanulate flowers composed of 6 basally fused tepals, shaped into a flared trumpet.
   Length.—Approximately 2.5 cm.
   Width.—Approximately 2.75 cm at widest point.
   Lobe length.—Approximately 1.5 cm.
   Lobe width.—Average 0.7 cm.
   Margin.—Entire.
   Shape.—Lanceolate.
   Apex.—Rounded with very shallow, irregular notches.
   Texture.—Glabrous all surfaces.
   Color.—Tepals: When opening, upper surface: Near RHS White 155C. When opening, lower surface: Near RHS White 155B. Fully opened, upper surface: Near RHS White 155D. Fully opened, lower surface: Near RHS White 155D.
Perianth tube:
   Length.—1 cm.
   Color.—Inner surface: Near RHS White 155D. Outer surface: Near RHS White 155D.
Fragrance: Faint musty scent.
Sepals: Not present.
Pedicel:
   Length.—1.7 to 2.2 cm.
   Texture.—Glabrous.
   Color.—Near RHS Green 143C.

### REPRODUCTIVE ORGANS

Stamens:
   Protrusion.—No protrusion past the floral lobe apex.
   Filament color.—Near RHS White 155D.
Anthers:
   Quantity.—6.
   Length.—Approximately 0.3 cm.
   Color.—Near RHS Yellow-White 158A.
Pistil:
   Number.—1.
   Length.—Approximately 1.5 cm.
   Style.—Color: Near RHS White 155D.
   Stigma.—Shape: Linear. Color: Near RHS White 155D.

### OTHER CHARACTERISTICS

Seeds: Not observed.
Disease/pest resistance: Neither resistance no susceptibility to normal diseases and pests of Agapanthus.
Temperature tolerance: Not tested but expected to tolerate −5° C. to about 30° C.
Drought tolerance: Will tolerate considerable dryness once established.

What is claimed is:

1. A new and distinct cultivar of Agapanthus plant named ‘MONSAPHO’ as herein illustrated and described.
